(19) United States
(12) Patent Application Publication
(10) Pub. No.: US 2012/0258922 A1
(43) Pub. Date: Oct. 11, 2012

(54) SYNERGISTIC INHIBITION OF LOW-DENSITY LIPOPROTEIN OXIDATION

(75) Inventor: Sekhar Boddupalli, Sacramento, CA (US)

(73) Assignee: Seminis Vegetable Seeds, Inc.

(21) Appl. No.: 13/441,507

(22) Filed: Apr. 6, 2012

Related U.S. Application Data
(60) Provisional application No. 61/473,604, filed on Apr. 8, 2011.

(51) Int. Cl. A61K 31/7028; A61P 7/00; A01H 5/00; A61K 31/355

(52) U.S. Cl. 514/24; 514/458; 800/306

(57) ABSTRACT

The invention provides use of a composition comprising a plant, plant parts, and extracts thereof having high levels of glucoraphanin and/or sulforaphane and tocopherols. Also provided are glucoraphanin and/or sulforaphane, in combination with tocopherols, where the overall composition synergistically inhibits oxidation of low-density lipoproteins. Methods of producing a food from such a plant are also provided.

### BACKGROUND OF THE INVENTION

This application claims the priority of U.S. Provisional Appl. Ser. No. 61/473,604, filed Apr. 8, 2011, the entire disclosure of which is incorporated herein by reference.

1. Field of the Invention

The present invention relates generally to methods and compositions for inhibiting low-density lipoprotein oxidation. More particularly, it concerns methods and compositions for providing sulforaphane or a precursor thereof and tocopherol that synergistically inhibit the oxidation of low-density lipoproteins.

2. Description of Related Art

Central to normal physiological function is the reduction-oxidation state of the cell, and an abnormal oxidation state may underlie disease. For instance, oxidation of low-density lipoproteins has been associated with cardiovascular disease and cancer as well as other conditions and diseases. Methods and compositions for reducing oxidation of low-density lipoproteins would thus represent a significant advance in the art.

### SUMMARY OF THE INVENTION

In one aspect of the present invention is a cruciferous vegetable plant that comprises an elevated endogenous level of glucoraphanin and tocopherol that is at least about twice that found in a standard variety of the same species of cruciferous vegetable plant. In another aspect the cruciferous vegetable plant is broccoli.

In another aspect the plant comprises an endogenous level of glucoraphanin that is at least about three times that of said standard variety of the same species.

In still another aspect the plant comprises an endogenous level of tocopherol, including where the tocopherol is γ-tocopherol, α-tocopherol or both, that is at least about three times that of the standard variety of the same species.

In yet another aspect of the present invention, there is provided a method of reducing low-density lipoprotein oxidation, comprising providing in the diet of a subject the plant of a cruciferous vegetable that comprises an elevated endogenous level of glucoraphanin and tocopherol that is at least about twice that found in a standard variety of the same species of cruciferous vegetable or a part thereof, wherein oxidation of low-density lipoprotein is reduced in the subject. The plant further comprises an endogenous level of glucoraphanin that is at least about three times that of said standard variety of the same species, or alternatively, the plant comprises an endogenous level of tocopherol that is at least about three times that of said standard variety of the same species where the tocopherol is γ-tocopherol or α-tocopherol.

In one aspect of the present invention, there is provided a method of reducing low-density lipoprotein oxidation where the glutathione:glutathione disulfide (GSH:GSSG) ratio is increased in cells of a subject and further where redox poise is increased.

In another aspect of the present invention, a composition is provided for reducing low-density lipoprotein oxidation that comprises at least about 2 μM sulforaphane or a precursor thereof, including glucoraphanin, and at least about 12 mIU/L of tocopherol, and further, where the tocopherol is γ-tocopherol or α-tocopherol. In another aspect, the composition comprises at least about 18 mIU/L α-tocopherol, at least about 25 mIU/L α-tocopherol, at least about 4 μM or more sulforaphane, at least about 6 μM sulforaphane, or at least about 8 μM sulforaphane.

In still yet another aspect of the present invention, a method of producing a food or feed comprising obtaining a plant of a cruciferous vegetable species that comprises an elevated endogenous level of glucoraphanin and tocopherol that is at least about twice that found in a standard variety of the same species, and producing food or feed from said plant or a part thereof. In one aspect, the plant is broccoli.

In still yet another aspect of the present invention, the standard variety of broccoli is the Marathon cultivar.

In still yet another aspect of the present invention, plants and compositions derived therefrom are provided that activate the human Nrf2 transcription factor at a concentration providing a level of activity similar to, for example, amounts of sulforaphane shown herein. This includes about 2-4 μM, including, in certain embodiments, about 1.5 μM, 2 μM, 2.5 μM, 3 μM and 3.5 μM. In specific embodiments, compositions and/or plant or products derived therefrom are provided comprising plant phenylpropenoids, e.g., cinnamic acids and their esters, phenylpropanones such as (10)-shogaol from ginger, chalcones such as isoliquiritigenin from licorice, curcuminoids such as curcumin from turmeric, coumarins, diterpenes such as carnisol from rosemary, and flavonoids such as quercetin from onion. See, e.g., Zoete et al., Free Radical Biology and Medicine, 36 (11), 1418-1423 (2004); Dinkova-Kostova and Talalay, Mol. Nutr. Food Res., 52, S128-S138 (2008); and Eggler et al., Mol. Nutr. Food Res., 52, S84-S94 (2008); each incorporated herein by reference in their entirety. Such compositions may be used, in particular embodiments, that have an effect on Nrf2 activation similar to sulforaphane. In one specific embodiment, such compositions may be used together with or in place of sulforaphane in a composition of the invention to reduce LDL oxidation. Such compositions may thus, for example, provide further methods for reducing LDL oxidation and/or prevent a cardiovascular disorder as outlined herein.

Embodiments discussed in the context of methods and/or compositions of the invention may be employed with respect to any other method or composition described herein. Thus, an embodiment pertaining to one method or composition may be applied to other methods and compositions of the invention as well.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation in a mixture or resulting from purification, variation arising from error for a device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein. In the figures, α-tocopherol is alternatively referred to as “A”, “AT” or “VIT E”. Sulforaphane is alternatively referred to as “S” or “SF”.

FIG. 1 is a graph showing inhibition of low-density lipoprotein (LDL) oxidation (referred to as “Ox- LDL” in FIG. 1) with increasing amounts of α-tocopherol on human monocyte-derived macrophages (HMDM) treated with 100 μg/ml LDL compared to vehicle control (referred to as “cells”) and LDL only (referred to as “LDL”). Along the x-axis, “A” refers to α-tocopherol and the number following the A is the micromolar concentration. For example, “A6.25” refers to cells treated with 6.25 μM α-tocopherol. * p<0.01 compared to LDL control.

FIG. 2 is a graph showing inhibition of LDL oxidation with varying amounts of sulforaphane on HMDM treated with 100 μg/ml LDL compared to vehicle control and LDL alone. As an example, “S0.1” refers to cells treated with 0.1 μM sulforaphane. * p<0.01 compared to LDL control.

FIG. 3 is a graph showing inhibition of LDL oxidation with sulforaphane, α-tocopherol, or both, on HMDM treated with 100 μg/ml LDL compared to vehicle control and LDL only. The sulforaphane treatment groups received 5 μM sulforaphane. As an example, “Vit E 25” refers to cells treated with 25 μM α-tocopherol.

FIG. 4 is a graph showing inhibition of LDL oxidation with 5 μM sulforaphane and varying concentrations of α-tocopherol on HMDM treated with 100 μg/ml LDL compared to vehicle control and LDL only. As examples, “VitE25” refers to cells treated with 25 μM α-tocopherol, and “VitE50,S5” refers to cells treated with 25 μM α-tocopherol and 5 μM sulforaphane. * p<0.01 compared to LDL control. #p<0.01 compared to either α-tocopherol or sulforaphane alone.

FIG. 5 is a dose-response curve showing the effect of 5 μM sulforaphane on the inhibition of oxidized LDL of HMDM with varying concentrations of α-tocopherol.

FIG. 6 is a graph showing inhibition of LDL oxidation with 2 μM sulforaphane and varying concentrations of α-tocopherol on HMDM treated with 100 μg/ml LDL compared to vehicle control and LDL only. As an example, “A25” refers to cells treated with 25 μM α-tocopherol, and “A50,S2” refers to cells treated with 50 μM α-tocopherol and 2 μM sulforaphane. * p<0.01 compared to LDL control. #p<0.01 compared to either α-tocopherol or sulforaphane alone.

FIG. 7 is a dose-response curve showing the effect of 2 μM sulforaphane on the inhibition of oxidized LDL of HMDM with varying concentrations of α-tocopherol.

FIG. 8 is a graph showing the effect of 2 μM sulforaphane on the glutathione:glutathione disulfide (GSH:GSSG) ratio with varying concentrations of α-tocopherol. * p<0.01 compared to LDL control. #p<0.01 compared to α-tocopherol or sulforaphane alone.

FIG. 9 is a graph showing the effect of varying concentrations of sulforaphane on the GSH:GSSG ratio with varying concentrations of α-tocopherol.

FIGS. 10A-B depict the effect of 2 μM sulforaphane on induction of quinone reductase and overall inhibition of oxidized LDL of HMDM with varying concentrations of α-tocopherol. (A) is a graph showing the effect of 2 μM sulforaphane and various concentrations of α-tocopherol. (B) shows the fold induction of quinone reductase relative to the effects of LDL alone.

FIG. 11 is a graph comparing the inhibition of LDL oxidation with broccoli extracts, 2 μM sulforaphane, and 25 μM α-tocopherol on HMDM treated with 100 μg/ml LDL compared to vehicle control and LDL only. Concentrations are micromolar. For example, “A25” refers to cells treated with 25 μM α-tocopherol, and “A50,S2” refers to cells treated with 50 μM α-tocopherol and 2 μM sulforaphane.

FIG. 12 is a graph showing the effect of prior cell transfection with siRNA for silencing the Nrf2 transcription factor and its effect on inhibition of LDL oxidation with 2 μM sulforaphane (S2) and a fixed 25 μM concentration of α-tocopherol (A25) in HMDM treated with 100 μg/ml LDL compared to either treatment alone or to a non-functional, sequence scrambled siRNA (labeled “Scrambled si”).

### DESCRIPTION OF ILLUSTRATIVE EMBODIMENTS

The present invention relates to plants and parts thereof having improved nutritional characteristics providing health benefits. More particularly, the present invention relates to plants, including plant parts, which contain levels of tocopherol and sulforaphane compounds or precursors thereof that surprisingly yield a synergistic reduction in the oxidation of low-density lipoprotein and the oxidation state of a cell.

In one aspect of the present invention plants and compositions derived therefrom are provided having elevated levels of glucoraphanin and tocopherol that provide, for example, cardiovascular health benefits when consumed in the diet of a subject. The sulforaphane precursor, glucoraphanin, is metabolized to sulforaphane during consumption of food containing glucoraphanin by mammals such as humans. Cruciferous vegetable plants, including members of Brassicaceae, and more specifically broccoli, are particularly suitable for having elevated levels of glucoraphanin and/or sulforaphane, as well as tocopherol, in accordance with the invention.

Plants according to the invention or parts thereof may be provided with an elevated glucoraphanin and/or sulforaphane and tocopherol content that is at least twice the level found in standard plants grown under similar conditions. Elevated levels of glucoraphanin, for example, include amounts of at least about 10 μmol/g dry weight, at least about 14 μmol/g, at least about 16 μmol/g, at least about 20 μmol/g, at least about 25 μmol/g, at least about 30 μmol/g, at least about 50 μmol/g, or at least about 75 μmol/g, or more of the respective plant and/or plant part provided in the diet of a subject. As described in U.S. Patent Pub. No. 20110055945, incorporated by reference herein in its entirety, broccoli plant parts, namely florets, may be engineered to exhibit elevated levels of glucoraphanin of, for example, about 10 μmol/g, about 15 μmol/g, about 20 μmol/g, about 25 μmol/g, about 30 μmol/g, about 40 μmol/g, about 50 μmol/g, about 60 μmol/g, about 70 μmol/g, about 80 μmol/g, about 90 μmol/g, about 100 μmol/g, about 125 μmol/g, or at least about 150 μmol/g per dry weight or more.

In certain embodiments of the invention, the elevated level of tocopherol is an amount of at least about 0.25 mg/100 g, at least about 0.50 mg/100 g, at least about 1 mg/100 g, at least about 1.5 mg/100 g, at least about 2 mg/100 g, at least about 2.5 mg/100 g, at least about 3 mg/100 g, at least about 3.5 mg/100 g, at least about 4 mg/100 g, at least about 5 mg/100 g, at least about 6 mg/100 g, at least about 7 mg/100 g, at least about 8 mg/100 g, at least about 9 mg/100 g, at least about 10 mg/100 g, at least about 11 mg/100 g, at least about 12 mg/100 g, at least about 14 mg/100 g, at least about 16 mg/100 g, at least about 18 mg/100 g, at least about 20 mg/100 g, or at least about 25 mg/100 g or greater of the fresh weight of the plant or part thereof being measured.

In another embodiment, the elevated levels of tocopherol is an amount of at least about 0.38 IU/100 g, at least about 0.75 IU/100 g, at least about 1.5 IU/100 g, at least about 2.25 IU/100 g, at least about 3.0 IU/100 g, at least about 3.75 IU/100 g, at least about 4.5 IU/100 g, at least about 5.25 IU/100 g, at least about 6 IU/100 g, at least about 7.5 IU/100 g, at least about 9 IU/100 g, at least about 10.5 IU/100 g, at least about 12 IU/100 g, at least about 13.5 IU/100 g, at least about 15 IU/100 g, at least about 16 IU/100 g, or greater per fresh weight of the plant or part thereof being measured.

Plants according to the invention or parts thereof having elevated levels of glucoraphanin and/or sulforaphane are further provided wherein, in specific embodiments, the ratio of glucoraphanin to tocopherol is greater than about 5:1, greater than about 50:1, greater than about 100:1, greater than about 150:1, greater than about 200:1, greater than about 250:1, greater than about 500:1, greater than about 1000:1, greater than about 1400:1, or greater.

There are also provided herein methods of use of a composition comprising a plant or part thereof having elevated levels of glucoraphanin and/or sulforaphane and tocopherol. In such methods, the plant may be a Cruciferous vegetable including a member of Brassicaceae, and broccoli in particular. In such methods, a composition may be used having elevated levels of glucoraphanin and/or sulforaphane and tocopherol that comprises, for example, the florets, inflorescences, stalks, stems, or leaves of a plant provided herein. Cruciferous vegetables from the family Brassicaceae provided herein to contain elevated levels of glucoraphanin and/or sulforaphane and tocopherol include broccoli, watercress, cauliflower, kale, turnip, collards, Brussels sprouts, cabbage, and radish.

Extracts from the plants according to the invention or parts thereof having elevated levels of glucoraphanin and/or sulforaphane and tocopherol are also provided herein that may also be used in the practice of the present invention.

In specific embodiments of the invention, plants or parts thereof are provided to a subject so as to deliver glucoraphanin and/or sulforaphane in amounts of at least about 20 μmol/day, at least about 30 μmol/day, at least about 40 μmol/day, at least about 50 μmol/day, at least about 60-140 μmol/day, at least about 70-140 μmol/day, at least about 75-140 μmol/day, at least about 80-135 μmol/day, at least about 90-135 μmol/day, at least about 140 μmol/day, at least about 180 i.tmol/day, at least about 200 μmol/day, at least about 250 μmol/day, at least about 300 μmol/day, or greater of glucoraphanin and/or sulforaphane. Plants of the present invention and parts thereof may be consumed to provide, for example, a sulforaphane plasma concentration of at least about 0.05 μM, at least about 0.1 μM, at least about 0.5 μM, at least about 1.0 μM, at least about 1.5 μM, at least about 2.0 μM, at least about 2.5 μM, at least about 3.0 μM, at least about 3.5 μM, at least about 4.0 μM, at least about 4.5 μM, at least about 5.0 μM, at least about 5.5 μM, at least about 6.0 μM, at least about 6.5 μM, at least about 7.0 μM, at least about 7.5 μM, at least about 8.0 μM, at least about 8.5 μM, at least about 9.0 μM, at least about 9.5 μM, at least about 10.0 μM, at least about 12 μM, at least about 14 μM, or greater. Similarly, the plants of the invention and the parts thereof may be consumed in an amount to provide, for example, a tocopherol plasma concentration of at least about 0.5 μM, at least about 0.8 μM, at least about li.tM, at least about 2 μM, at least about 3 μM, at least about 4 μM, at least about 5 μM, at least about 10 μM, at least about 15 μM, at least about 20 μM, at least about 25 μM, at least about 30 μM, at least about 35 μM, at least about 40 μM, at least about 50 μM, or at least about 55 μM, or greater. It is also contemplated that the plants of the present invention and parts thereof may be consumed such that elevated levels of glucoraphanin and/or sulforaphane and tocopherol to provide a relative plasma concentration of glucoraphanin and/or sulforaphane to tocopherol of about 1:40, about 1:30, about 1:20, about 1:10, about 1:9, about 1:8, about 1:7, about 1:6, about 1:5, about 1:4, about 1:3, about 1:2, about 1:1, about 2:3, about 4:5, or greater.

A method for the treatment or prevention of a cardiovascular disorder is provided comprising administering to a subject an effective amount of a plant of the present invention or part thereof having elevated levels of glucoraphanin and/or sulforaphane and tocopherol. In particular, the composition may be used in managing the cardiovascular health or cardiovascular disease progression or enhancing cardiovascular health and wellness of a person, mammal, or animal. The compositions may be used to improve or control the level of mammalian serum cholesterol, such as the level of total cholesterol (TC) and/or the level of low density lipoprotein-cholesterol (LDL-cholesterol), and/or the inflammatory and atherogenic potential of either TC or LDL-cholesterol. Similarly, a composition comprising plants of the present invention and parts thereof having an elevated level of glucoraphanin and/or sulforaphane and tocopherol may be used in the treatment or prevention of cardiovascular inflammation and/or the treatment or prevention of angina, atherosclerosis, cardiomyopathy or cardiac inflammation, congestive heart failure, coronary artery disease, carotid artery disease, coronary thrombosis, myocardial infarction, hypertension, hyperlipidemia, hypercholesterolemia, peripheral artery disease, and stroke.

The present invention further provides methods and compositions for inhibiting low- density lipoprotein (LDL) oxidation in general, and using sulforaphane and tocopherol to synergistically inhibit LDL oxidation in particular.

Furthermore, plants of the present invention may be produced as food or feed having the ratios of sulforaphane to tocopherol that synergistically inhibit LDL oxidation described previously.

### Definitions

“Glucoraphanin” refers to 4-methyl-sulfinyl-butyl glucosinolate and is also abbreviated “MSB” herein.

The phrase “elevated level” or “elevated levels” of glucoraphanin and/or sulforaphane refers to a content at least twice the level of glucoraphanin and/or sulforaphane on a w/w basis where the level is the amount found in a standard plant of the same species, or a corresponding part thereof, grown under similar conditions. In the case of broccoli, glucoraphanin content is about 1 μg/g dry weight in stems and about 3 mg/g dry weight for inflorescences.

The phrase “elevated level of tocopherol” means the plants according to the present invention, such as a Cruciferous vegetable, and parts thereof, have a level of tocopherol that is at least twice the level of tocopherol found in a standard plant or their respective plant part grown under similar conditions, except where specific levels are provided, such as the broccoli content of α-tocopherol and γ-tocopherol, that follow. In specific embodiments of the invention, an “elevated level of tocopherol” in broccoli, for example, means broccoli that contains levels of α-tocopherol that is at least about at least about 4.3 mg/100 g, at least about 4.5 mg/100 g, at least about 4.75 mg/100 g, at least about 5 mg/100 g, at least about 6 mg/100 g, at least about 7 mg/100 g, at least about 8 mg/100 g, at least about 9 mg/100 g, at least about 10 mg/100 g, at least about 11 mg/100 g, at least about 12 mg/100 g, at least about 14 mg/100 g, at least about 16 mg/100 g, at least about 18 mg/100 g, at least about 20 mg/100 g, at least about 25 mg/100 g, or more.

“Standard”, regarding broccoli, refers to broccoli inflorescences containing 4.29 mg/100 g α-tocopherol or less and/or 0.64 mg/100 g γ-tocopherol or less per fresh weight, and glucoraphanin of about 5.5 micromol/g or less dry weight. “Standard”, in referring to cauliflower or Brussels sprouts, refers to 1.2 mg/100 g α-tocopherol or less. “Standard”, in referring to cabbage, refers to 0.27 mg/100 g α-tocopherol or less. “Standard”, in referring to kale, refers to 2.8 mg/100 g α-tocopherol or less and/or 0.08 mg/100 g γ-tocopherol or less per fresh weight for other Cruciferous vegetables.

“Tocopherol” as used herein refers to the compounds that include α, β, γ, δ-tocopherols and α, β, γ, and δ-tocotrienols, and refers to one or more of the various forms. For example, as used herein, “tocopherol” may refer to α-tocopherol alone or a combination of α- and γ-tocopherols, etc.

The term “fresh Cruciferous vegetable” as used herein means a Cruciferous vegetable or part thereof either consumed raw or cooked by any suitable method.

The term “processed Cruciferous vegetable” as used herein means a Cruciferous vegetable that has undergone at least one further processing step such as, for example, floreting, individual quick freezing, maceration, homogenization, drying, freezing, compacting, etc.

An “extract” as used herein refers to a substance or mixture of substances obtained by extracting the whole or part of a fresh Cruciferous vegetables as defined herein and/or by extracting the whole or part of a processed Cruciferous vegetables as defined herein. The extraction may be carried out using a solvent such as ethanol or water. In one embodiment the extract is an aqueous extract. In one embodiment the extract comprises glucoraphanin and/or sulforaphane and tocopherol from a starting Cruciferous vegetable.

The term “fresh broccoli” as used herein means uncooked broccoli inflorescences and stems that have substantially retained the nutritional content at the time of harvest without, for example, being subject to rotting.

The terms “administer”, “administering”, “treating” or “treated” used herein include making available for consumption adequate quantities of elevated glucoraphanin and/or sulforaphane and tocopherol in a Cruciferous vegetable, part thereof, or composition therefrom.

The term “mammal,” as used herein, refers to any animal classified as a mammal, including humans.

As used herein, the term “inhibition” means to decrease a concentration of a biological substance, wherein such inhibition may be achieved by any of the biological mechanisms, such as, e.g., inhibition of synthesis or activity of the biological substance.

The term “synergy” and “synergistically” refer to the combined action between two or more compounds that is greater than merely additive or greater than expected based on bioassay of individual components.

As used herein, an “effective amount” means the dose or amount to be administered to a subject, and the frequency of administration to the subject, which are readily determined by one of ordinary skill in the art by the use of known techniques and by observing results obtained under analogous circumstances to effectively treat a disease or provide a health benefit given the principles elucidated herein. In determining the effective amount or dose, a number of factors are considered by an attending diagnostician, including but not limited to, the potency and duration of action of the compounds used; the nature of the condition to be treated as well as the sex, age, weight, general health and individual responsiveness of the subject to be treated; and other relevant circumstances known to one skilled in the art.

The term “effective” indicates the amount of an agent to cause a change in the value to be measured, or improve the condition or state of the cell or organism. The term “effective” is to be understood to be equivalent to the phrase “effective for the change in condition,” and both are intended to qualify, e.g., the amount of sulforaphane and tocopherol used in the methods of the present invention which will achieve the goal of reducing LDL oxidation or reducing any health condition resulting therefrom.

Plants having elevated levels of glucoraphanin and/or sulforaphane and tocopherol of the present invention may be prepared following methods generally known in the art, e.g., as described in U.S. Pat. Nos. 7,230,165; 6,977,297; 6,340,784, and U.S. Patent Pub. No. 20110055945, each herein incorporated by reference in its entirety. For example, up-regulation of glucoraphanin via breeding techniques is described in U.S. Patent Pub. No. 20110055945, in which broccoli hybrids were produced having glucoraphanin levels in stalks and florets over twice the corresponding levels observed in other varieties. Similarly, plants with elevated tocopherol expression may be produced according to methods previously described. For example, in U.S. Pat. No. 7,230,165, incorporated herein in its entirety, transgenic plants engineered to over-express the gene encoding phytol kinase, one of the enzymes in the tocopherol biosynthetic pathway, exhibited significantly elevated tocopherol levels in plants and seeds.

Other features, objects and advantages of the present invention will be apparent to those skilled in the art. The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the present invention.

All publications and patent applications cited in this specification are herein incorporated by reference as if each individual publication or patent application was specifically and individually indicated to be incorporated by reference.

### EXAMPLE 1

Blood was drawn and collected from healthy human volunteers, and LDL was isolated by density gradient centrifugation following methods known to one of ordinary skill in the art (e.g., Napolitano et al., Int J Biochem Cell Biol, 35:1127-43, 2002). Monocytes were also isolated from the blood and separated by Ficoll Hypaque centrifugation also according to known methods (e.g., Napolitano et al., Eur J Clin Invest, 35:482-90, 2005), followed by negative magnetic separation and maintained in culture with 20% autologous serum or F-10 medium for 5-7 days to differentiate into human monocyte-derived macrophages (HMDM). HMDM served as the platform to examine macrophage-mediated LDL oxidation and to test the antioxidant efficacy of sulforaphane (SF), α-tocopherol (“AT” or “A”), or both. HMDM cells were pre-incubated for 12 hours with varying concentrations of sulforaphane alone, α-tocopherol alone, sulforaphane and α-tocopherol. Following the pre-incubation step, LDL (100 μg/ml) was added and the cells were incubated for 24 hours. Supernatants were used for assessing oxidized LDL by ELISA (Mercodia) and/or TBARS (“Thiobarbituric Acid Reactive Substances”) as described previously (Devaraj et al., J Clin Invest 98:756, 1996). To assess the glutathione:glutathione disulfide (GSH:GSSG) ratio as an indicator of the redox poise of the cell, cells were dislodged by PBS-EDTA and were lysed (Pierce MPER) after a freeze-thaw cycle. After sonication and centrifugation, the cell pellet was stored at −80° C. until the relative levels of GSH and GSSG were assayed (Oxis Biochemicals). Quinone reductase activity was assayed following the method described previously (J. Fahey et al., Methods in Enzymology 382, 243-258, 2004).

Raw broccoli extracts (“BE” or “B. Extract”) were prepared from four hybrids. BE 2204 and BE 2206 are commercially-sold standard broccoli while BE 2144 and BE 2244 are broccoli hybrids with 2-3× higher glucoraphanin compared BE 2204 and BE 2206. 50 grams of extract were mixed with equal parts 50mM MES buffer pH 6.0 solution. Samples were blended for approximately 1 minute until homogeneous then maintained at room temperature for 10 min. Filtrates from samples were diluted 1:40 with 50 mM MES buffer for the assay. Final diluted sulforaphane concentrations in the extracts ranged from 1 μM to 5 μM and were determined per hybrid as follows: BE 2204 =2.49 μM, BE 2208=1.13 μM, BE 2144=3.44 μM, and BE 2244=5.67 μM. Assays with the broccoli extracts were performed as described above.

### EXAMPLE 2

### Sulforaphane and α-Tocopherol Individually Inhibit LDL Oxidation

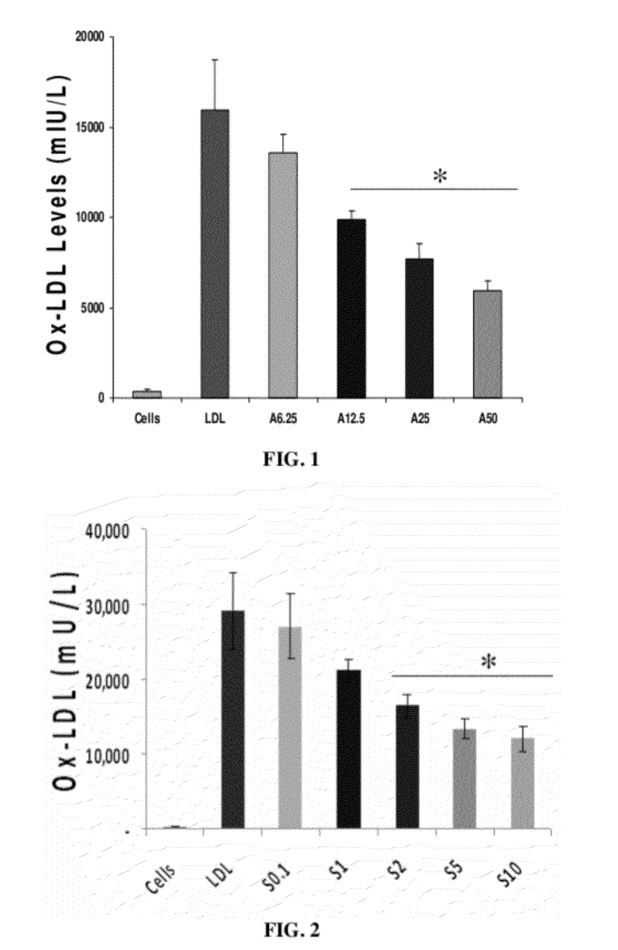

Sulforaphane and α-tocopherol individually inhibit LDL oxidation. Each sulforaphane and α-tocopherol individually showed a dose-dependent inhibition of LDL oxidation with a maximum inhibition of 58% compared to the LDL control. (FIG. 1; FIG. 2) α-Tocopherol at concentrations greater than 12.5 μM significantly inhibit the oxidation of LDL (Ox-LDL) and higher concentrations elicit stronger inhibition. (FIG. 1). Similarly, sulforaphane at concentrations greater than 2 μM significantly inhibit the oxidation of LDL with higher concentrations, for example, 5 μM and 10 μM, eliciting stronger inhibition. (FIG. 2).

### EXAMPLE 3

### Sulforaphane and α-Tocopherol Synergistically Inhibit LDL Oxidation

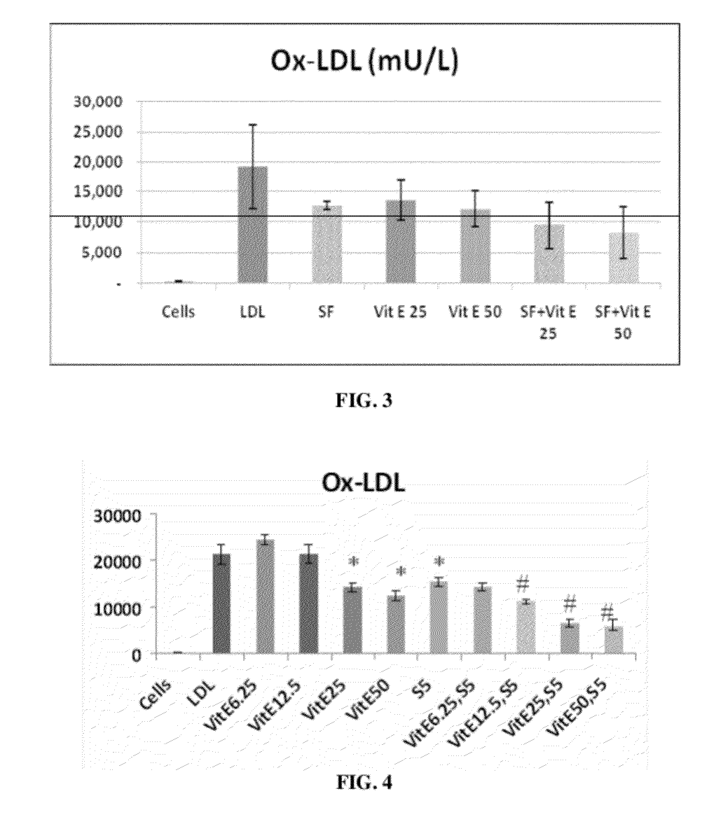

It was surprisingly found, as illustrated in FIG. 3, that sulforaphane and α-tocopherol synergistically inhibited LDL Oxidation. For example, 5 μM sulforaphane in combination with increasing concentrations of α-tocopherol significantly inhibited the oxidation of LDL with progressively higher concentrations α-tocopherol eliciting greater inhibition with higher concentrations. (FIGS. 3 and 4). In particular, 5 μM sulforaphane with 25 μM and 50 μM α-tocopherol synergistically inhibit LDL oxidation compared to either α-tocopherol or sulforaphane alone. (FIG. 4). In fact, the combination of 5 μM sulforaphane and 25 μM α-tocopherol resulted in 78% inhibition of LDL oxidation.

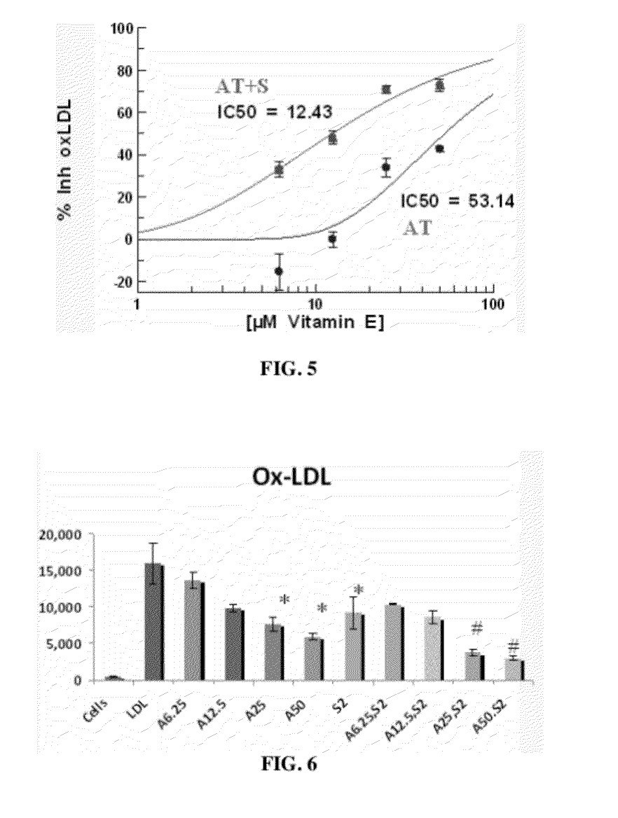

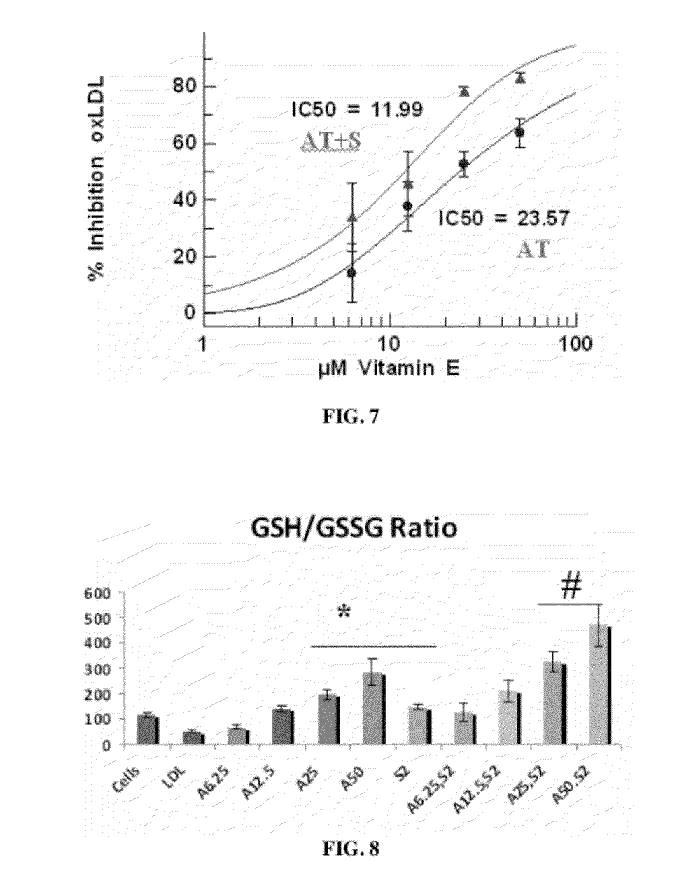

FIG. 5 is a dose-response curve of the data of FIG. 4. FIG. 5 demonstrates that treatment of the cells with 5 μM sulforaphane and α-tocopherol shifts the IC50 of LDL oxidation of α-tocopherol alone to 12.43 μM from 53.14 μM. (FIG. 5). Similarly, synergistic inhibition was also observed with 2 μM sulforaphane and increasing concentrations of α-tocopherol, (FIG. 6). In fact, 2 μM sulforaphane shifts the IC50 of LDL oxidation of α-tocopherol alone from 23.57 to 11.99 μM. (FIG. 7), indicating that a lower concentration of sulforaphane was sufficient to elicit synergistic inhibition with α-tocopherol.

### EXAMPLE 4

### Sulforaphane and α-Tocopherol Synergistically Improve Redox Poise

The combination of sulforaphane and α-tocopherol synergistically increased the redox poise of the cells. In particular, sulforaphane and α-tocopherol synergistically increase the GSH:GSSG ratio to counter the oxidative stress of the cell and reduce overall LDL oxidation. Supplementation of LDL alone depleted the GSH:GSSG ratio. (FIGS. 8, 9). Both sulforaphane and α-tocopherol individually restored the ratio to normal levels at lower doses. Surprisingly, however, a synergistic increase in the GSH:GSSG ratio was observed with the combination of 5 μM sulforaphane and 25 μM α-tocopherol (FIGS. 8, 9), resulting in a decreased oxidation state for the cell and an improved overall redox poise.

### EXAMPLE 5

### Sulforaphane and α-Tocopherol Synergistically Induce Quinone Reductase

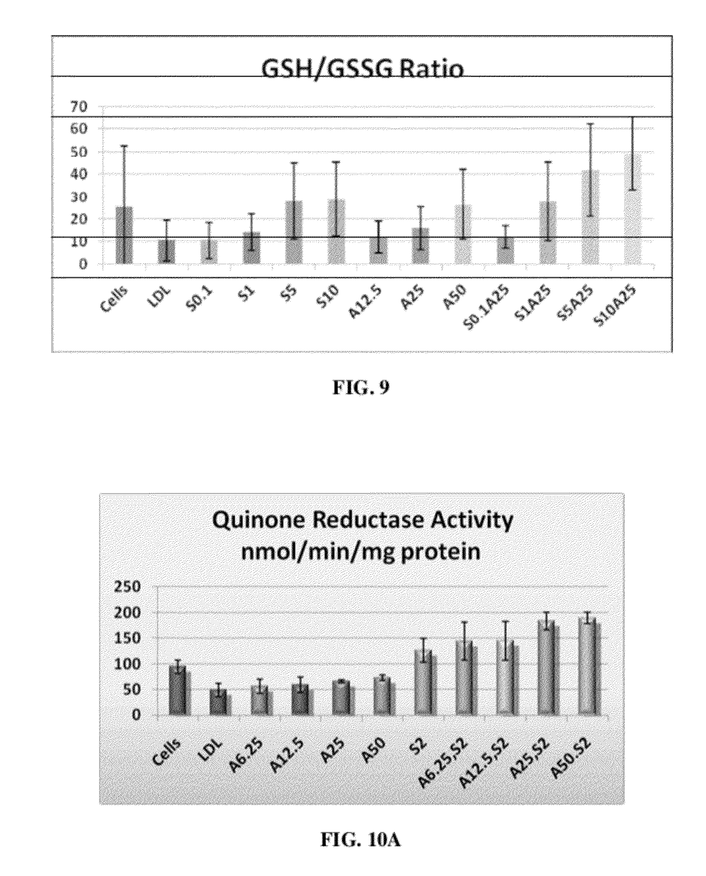

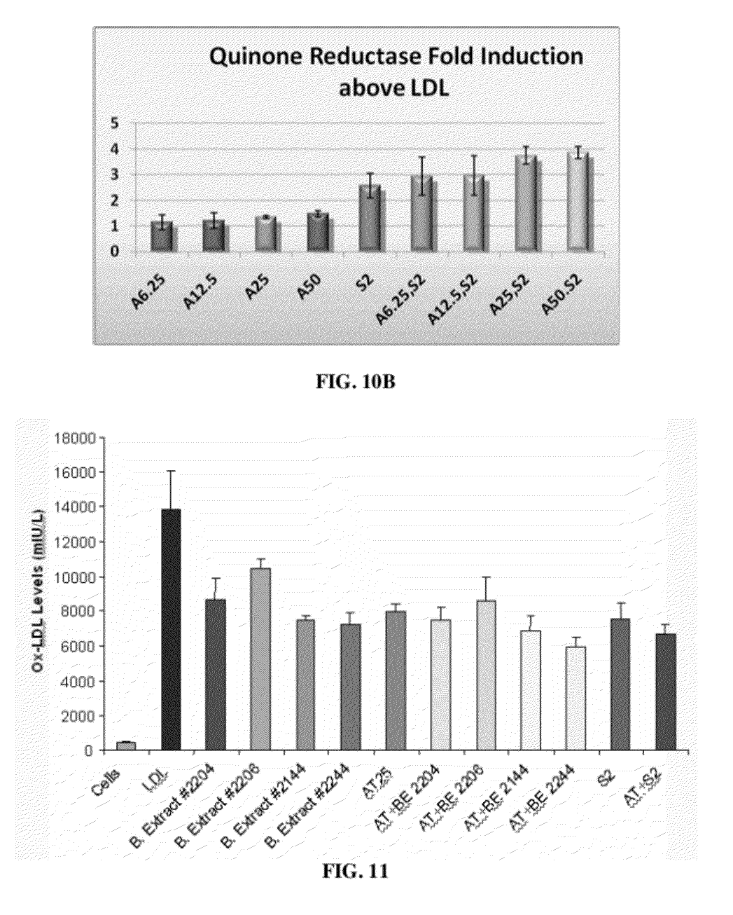

The combination of sulforaphane and α-tocopherol synergistically increased quinone reductase activity of the cells. Supplementation of LDL reduced quinone reductase activity in the absence of sulforaphane and α-tocopherol as shown in Table 1. However, sulforaphane individually restored enzymatic activity to normal levels while and α-tocopherol had only a modest effect individually. (Table 1 and FIGS. 10A,B). Surprisingly, synergetic induction of quinone reductase was observed with the combination of 2 μM sulforaphane and with 6.25 μM α-tocopherol and higher concentrations (FIGS. 10A, 10B), resulting in a decreased oxidation state in the cell.

TABLE 1
Inducement of quinone reductase activity with
sulforaphane and α-tocopherol.
QUINONE REDUCTASE ACTIVITY (nmol/min/mg protein).

| Treatment | Expt. 1 | Expt. 2 | Expt. 3 | Average |
|---|---|---|---|---|
| Untreated cells | 104.56 | 98.74 | 79.56 | 94.28667 |
| LDL | 58.79 | 54.12 | 34.56 | 49.15667 |
| A6.25 | 59.87 | 68.97 | 41.12 | 56.65333 |
| A12.5 | 66.45 | 69.47 | 42.25 | 59.39 |
| A25 | 68.98 | 64.56 | 65.14 | 66.22667 |
| A50 | 77.14 | 74.19 | 66.23 | 72.52 |
| S2 | 151.23 | 124.23 | 104.56 | 126.6733 |
| A6.25, S2 | 184.25 | 135.64 | 112.58 | 144.1567 |
| A12.5, S2 | 186.65 | 136.69 | 112.74 | 145.36 |
| A25, S2 | 199.14 | 187.45 | 165.45 | 184.0133 |
| A50, S2 | 199.97 | 191.25 | 178.29 | 189.8367 |

### EXAMPLE 6

### Broccoli Extracts Alone and in Combination with α-Tocopherol Inhibit LDL Oxidation

These beneficial effects of sulforaphane and α-tocopherol were mimicked by broccoli extracts. (FIG. 10). Cells were treated with broccoli extracts from four different hybrids and all were found to inhibit LDL oxidation. The broccoli extracts were added in combination with α-tocopherol and hybrid extract 2244 which created the highest sulforaphane concentration (5.67 μM), in combination with 25 μM alpha-tocopherol, provided statistically lower oxidized LDL compared to 25 μM alpha-tocopherol alone. Sulforaphane (2 μM) and α-tocopherol (25 μM) individually applied to the cells also reduced LDL oxidation as observed previously, thus confirming the effect.

### EXAMPLE 7

### The Synergistic Inhibition of LDL Oxidation by Sulforaphane and α-tocopherol Acts Through the Nrf2 Pathway

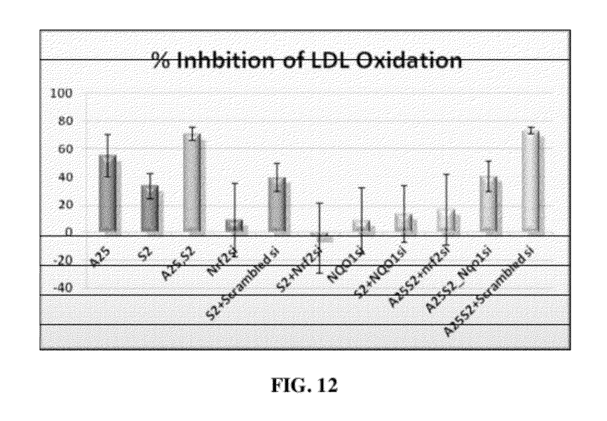

A study was carried out to determine the effect of silencing the Nrf2 transcription factor on inhibition of LDL oxidation with 2 μM sulforaphane (S2) and a fixed 25 μM concentration of α-tocopherol (A25). Cells were transfected with siRNA for silencing the Nrf2 transcription factor, followed by measuring inhibition of LDL oxidation with 2 μM sulforaphane (S2) and a fixed 25 μM concentration of α-tocopherol (A25) in HMDM treated with 100 μg/ml LDL, compared to either treatment alone or to a non-functional, sequence scrambled siRNA (labeled “Scrambled si”). The results are show in FIG. 12 and indicate that the synergistic effect of sulforaphane on the activity of α-tocopherol at inhibiting LDL oxidation in HMDM can act through the Nrf2 antioxidant pathway, and may be blocked by siRNA silencing. A similar study carried out using SiNQO1, which silences the antioxidant phase 2 enzyme product of the Nrf2 transcription factor NQO1, showed reduced but not complete reduction of the effectiveness of α-tocopherol (FIG. 12).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### References

The references listed below are incorporated herein by reference to the extent that they supplement, explain, provide a background for, or teach methodology, techniques, and/or composition employed herein.

Fimognari et al., Curr Drug Metab, 9(7):668-78, 2008

Holvoet, Circulation, 98(15): 1487-1494, 1998

Holvoet, Arter, Thromb Vasc Biol, 21(5):844-848, 2001

Hulthe, Arter, Thromb Vasc Biol, 22(7):1162-1167, 2002

Jones D P et al., Free Radic Biol Med, 47(10):1329-38, 2009

Kurilich et al., J Agric Food Chem, 47:1576-1581, 1999

Napolitano et al., Int J Biochem Cell Biol, 35: 1127-43, 2002

Napolitano et al., Eur J Clin Invest, 35: 482-90, 2005

Nishi, Arter, Thromb Vasc Biol, 22(10):1649-1654, 2002

Saremi et al., Am J Ther, 17(3):e56-65, 2010

Yoshida et al., Clin Chim Acta, 411(23-24):1875-82, 2010

What is claimed is:

1. A plant of a cruciferous vegetable species, wherein the plant comprises an elevated endogenous level of glucoraphanin and tocopherol that is at least about twice that found in a standard variety of the same species as said cruciferous vegetable

2. The plant of claim 1, wherein the cruciferous vegetable species is broccoli.

3. The plant of claim 1, comprising an endogenous level of glucoraphanin that is at least about three times that of said standard variety of the same species.

4. The plant of claim 1, comprising an endogenous level of tocopherol that is at least about three times that of said standard variety of the same species.

5. The plant of claim 1, wherein the tocopherol is γ-tocopherol.

6. The plant of claim 1, wherein the tocopherol is α-tocopherol.

7. The plant of claim 1, wherein the tocopherol is γ-tocopherol and α-tocopherol.

8. A method of reducing low-density lipoprotein oxidation in a subject, comprising providing in the diet of the subject the plant of claim 1 or a part thereof, wherein oxidation of low-density lipoprotein is reduced in the subject.

9. The method of claim 8, wherein the plant of claim 1 comprises an endogenous level of glucoraphanin that is at least about three times that of said standard variety of the same species.

10. The method of claim 8, wherein the plant of claim 1 comprises an endogenous level of tocopherol that is at least about three times that of said standard variety of the same species.

11. The method of claim 8, wherein said tocopherol is γ-tocopherol.

12. The method of claim 8, wherein said tocopherol is α-tocopherol.

13. The method of claim 8, wherein the glutathione:glutathione disulfide (GSH:GSSG) ratio is increased in cells of said subject.

14. The method of claim 8, wherein the redox poise is increased in cells of said subject.

15. The method of claim 8, wherein the subject is a human.

16. The method of claim 8, wherein said cruciferous vegetable species is broccoli.

17. A composition for reducing low-density lipoprotein oxidation comprising at least about 2 μM sulforaphane or a precursor thereof and at least about 12 mIU/L of tocopherol.

18. The composition of claim 17, wherein said tocopherol is γ-tocopherol.

19. The composition of claim 17, wherein said tocopherol is α-tocopherol.

20. The composition of claim 17, wherein said composition comprises at least about 18 mIU/L α-tocopherol.

21. The composition of claim 17, wherein said composition comprises at least about 25 mIU/L α-tocopherol.

22. The composition of claim 17, wherein said composition comprises at least about 4 μM or more sulforaphane.

23. The composition of claim 17, wherein said composition comprises at least about 6 μM sulforaphane.

24. The composition of claim 17, wherein said composition comprises at least about 8 μM sulforaphane.

25. The composition of claim 17, wherein the precursor of sulforaphane is glucoraphanin.

26. A method of producing a food or feed comprising:
(a) obtaining a plant according to claim 1; and
(b) producing food or feed from said plant or a part thereof.

27. The method of claim 26, wherein said plant is a broccoli plant.
